(19) United States
(12) Patent Application Publication
(10) Pub. No.: US 2020/0054801 A1
(43) Pub. Date: Feb. 20, 2020

(54) Portable medical vacuum suction device

(71) Applicant: Hyun Woo LEE, Hanam-si (KR)

(72) Inventor: Hyun Woo LEE, Hanam-si (KR)

(21) Appl. No.: 16/072,528

(22) PCT Filed: Aug. 17, 2017
(86) PCT No.: PCT/KR2017/008987

§ 371 Date: Jul. 25, 2018

(30) Foreign Application Priority Data
Oct. 17, 2016 (KR) 10-2016-0134126
Mar. 24, 2017 (KR) 10-2017-0037367
Jul. 10, 2017 (KR) 10-2017-0087096

(51) Int. Cl. A61M 1/00

(52) U.S. Cl. CPC A61M 1/0031; A61M 1/0088; A61M 1/0082; A61M 1/0027; A61M 1/0003

(57) ABSTRACT

The present invention relates to a medical vacuum suction device used for forcibly discharging various body fluids and, more particularly, to a portable vacuum suction device comprising: a suction container provided with an internal space in a vacuum state; a connection cap coupled to an inlet of the suction container; a drain tube for guiding the discharge of a body fluid toward the connection cap; a flow rate control member for controlling the flow rate of the body fluid; and a soft corrugated tube which contracts or expands according to the vacuum pressure of the suction container.

### TECHNICAL FIELD

The present invention relates to a medical vacuum suction device used for forcibly discharging various body fluids and, more particularly, to a portable vacuum suction device including: a suction container provided with an internal space in a vacuum state; a connection cap coupled to an inlet of the suction container; a drain tube for guiding the discharge of a body fluid toward the connection cap; a flow rate control member for controlling the flow rate of the body fluid; and a soft corrugated tube which contracts or expands according to the vacuum pressure of the suction container.

### BACKGROUND ART

Generally, a medical suction device is a medical instrument for forcibly discharging a body fluid secreted from an operating portion of a patient or an airway of a patient in the intensive care unit, and the device includes a negative pressure providing means for providing a suction force, a storage container for storing the body fluid discharged by means of the suction force, and a drain tube inserted into the body of a human to induce the discharge of the body fluid.

A method for providing the negative pressure to the storage container in the medical suction device is divided into a manual method through manipulation of a piston by a user's hand, an elastic method using a returning force to an original state of a spring or an elastic container, and a vacuum method for in advance applying a vacuum pressure to the storage container.

One of conventional medical suction devices is disclosed in Korean Patent No. 10-0868360 (Dated on Nov. 5, 2008), wherein the medical suction device includes: a suction container made of a transparent synthetic resin material and having an internal space kept in a vacuum state; a suction pressure indication means located on one side of the suction container to indicate a suction pressure through a level of a suction pressure indication liquid; and a suction pressure fixing means for constantly maintaining the suction pressure of the suction container.

According to the above-mentioned conventional medical suction device, a vacuum force in the suction container can be constantly maintained through the suction pressure fixing means, and even if a momentary suction pressure is applied, the suction pressure can be accurately indicated. However, there is no means for arbitrarily controlling an amount of body fluid discharged by means of a user.

Also, a portable foreign substance removing device is disclosed in Korean Utility Model Registration No. 20-0449562 (Dated on Jul. 13, 2010), wherein the device includes: a storage container having a suction tube for sucking a foreign substance discharged from the body of a human and a discharge tube for discharging internal air therefrom; a vacuum motor for sucking the internal air of the storage container through the discharge tube of the storage container; and a body having a switch and a battery mounted therein to operate the vacuum motor.

According to the conventional foreign substance removing device, it is easy to use repeatedly and a given suction force can be kept for a long period of time. Due to the use of the vacuum motor, however, noise and vibrations are generated during the use, and the battery has to be also needed.

### PRIOR ART DOCUMENTS

(Patent document 1) Korean Patent No. 10-0868360 (Dated on Nov. 5, 2008)

(Patent document 2) Korean Patent No. 10-0948554 (Dated on Mar. 12, 2010)

(Patent document 3) Korean Utility Model Registration No. 20-0449562 (Dated on Jul. 13, 2010)

### DISCLOSURE

### Technical Problem

Accordingly, the present invention has been made in view of the above-mentioned problems occurring in the prior art, and it is an object of the present invention to provide a medical vacuum suction device used for forcibly discharging a body fluid by using a vacuum pressure in a suction container that is capable of checking whether an interior of the suction container is in a vacuum state with a user's naked eyes and capable of arbitrarily controlling an amount of body fluid discharged by the user, so that it can be portable and conveniently used.

### Technical Solution

To accomplish the above-mentioned object, according to the present invention, there is provided a portable medical vacuum suction device including: a suction container having an inlet formed on a top end periphery thereof and an internal space storing a body fluid discharged from the body of a human, the internal space being in a vacuum state; a connection cap sealedly coupled to the inlet of the suction container and having a body fluid inlet port protruding from one side thereof and an inside hole formed at the center thereof to connect the body fluid inlet port and the internal space of the suction container with each other; a drain tube coupled to the body fluid inlet port to induce the body fluid discharged from the body through a vacuum pressure of the suction container; a flow rate control member rotatably disposed in the inside hole of the connection cap to control a flow rate of the body fluid introduced into the suction container through the body fluid inlet port; and a soft corrugated tube disposed on one side of the suction container or the flow rate control member in such a manner as to contract or expand according to the vacuum pressure of the suction container.

### Advantageous Effects

According to the present invention, the medical vacuum suction device is used to forcibly discharge the body fluid by using a vacuum pressure in the suction container, which is portable, and also, the medical vacuum suction device is provided with the flow rate control member adapted to allow an amount of body fluid discharged to be arbitrarily controlled by the user and with the corrugated tube adapted to allow the vacuum state of the suction container to be checked with the user's naked eyes, which makes it convenient to use.

### DESCRIPTION OF DRAWINGS

FIG. 1 is a perspective view showing a medical vacuum suction device according to the present invention.

FIG. 2 is a sectional view showing the medical vacuum suction device according to the present invention.

FIG. 3 is a separate sectional view showing main operating parts of the medical vacuum suction device according to a first embodiment of the present invention.

FIG. 4 is a separate perspective view of FIG. 3.

FIG. 5 is a cross sectional view showing a flow rate control member of FIG. 3.

FIG. 6 is a separate sectional view showing main operating parts of the medical vacuum suction device according to a second embodiment of the present invention.

FIG. 7 is a separate perspective view of FIG. 6.

FIG. 8 is a sectional view showing the coupled state of FIG. 6.

<Explanation on reference numerals in the drawings>

| 10: suction container | 11: inlet |
|---|---|
| 12: exhaust valve | 13: transparent window |
| 14: outer toothed rim | 15: hanging loop |
| 20: connection cap | 21: body fluid inlet port |
| 22: inside hole | 23: inner toothed rim |
| 24: airtight member accommodation portion | |
| 25: connection port | |
| 30: drain tube | 31, 31a, 31b: luer connector |
| 40: flow rate control member | 41: cylinder |
| 42: handle | 43: center hole |
| 44, 44a, 44b: flow rate control groove | |
| 45: elastic slit | 46: circular locking projection |
| 47: passage cut-off wall | |
| 48: O-ring | 49: alignment protrusion |
| 50: corrugated tube | 51: support ring |
| 60: protection cap | 61: tube insertion slot |
| 70: fixing member | 71: insertion tube |
| 72: expanded wing | 80: airtight member |
| 81: cylinder insertion hole | |
| 82: body fluid passage | |

### BEST MODE FOR INVENTION

Hereinafter, the present invention will now be described in detail with reference to the attached drawings. Before the present invention is disclosed and described, it is to be understood that the disclosed embodiments are merely exemplary of the invention, which can be embodied in various forms. If it is determined that the detailed explanation on the well known technology related to the present invention makes the scope of the present invention not clear, the explanation will be avoided for the brevity of the description. Even though the corresponding parts in the embodiments of the present invention have somewhat different structures, the parts having the same functions as each other are indicated by corresponding reference numerals.

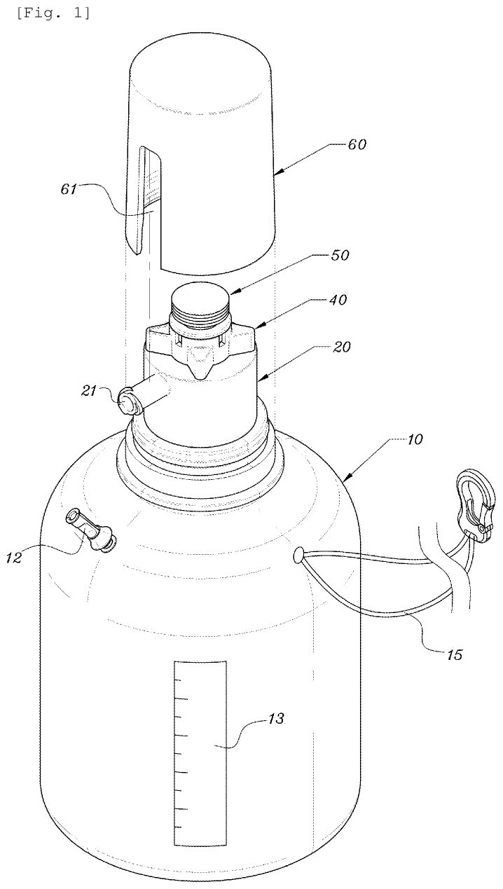

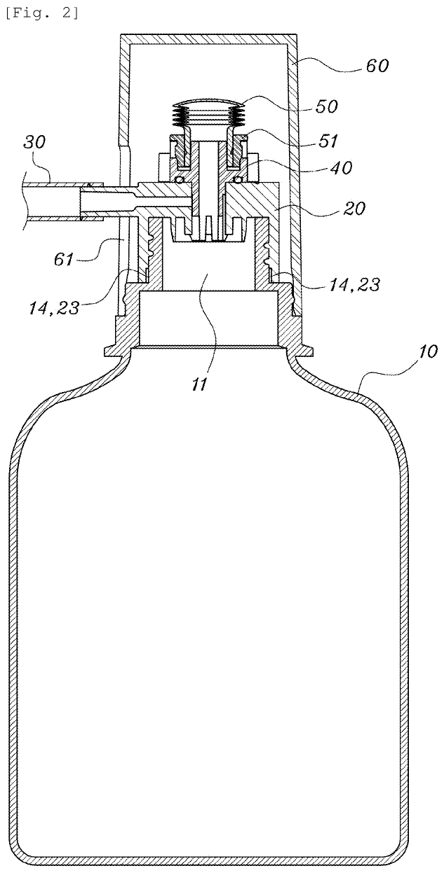

As shown in FIGS. 1 and 2, a portable medical vacuum suction device according to the present invention includes a suction container 10 provided with an internal space in a vacuum state, a connection cap 20 coupled to an inlet of the suction container 10, a drain tube 30 for guiding the discharge of a body fluid toward the connection cap 20, a flow rate control member 40 for controlling the flow rate of the body fluid, and a soft corrugated tube 50 which contracts or expands according to a vacuum pressure of the suction container 10.

Further, the portable medical vacuum suction device according to the present invention includes a protection cap 60 disposed on an upper end periphery of the suction container 10 to cover the connection cap 20, the flow rate control member 40 and the soft corrugated tube 50. The protection cap 60 has a tube insertion slot 61 adapted to pass the drain tube 30 therethrough, so that the protection cap 60 can be closed, without any inhibition of the drain tube 30.

First, the suction container 10 has an inlet 11 formed on a top end periphery thereof and the internal space for storing the body fluid discharged through the drain tube 30. Since the internal space of the suction container 10 is in the vacuum state, the suction container 10 has to have strength capable of maintaining the shape even under a vacuum pressure thereof.

Next, the connection cap 20 is sealedly coupled to the inlet 11 of the suction container 10 and has a body fluid inlet port 21 protruding from one side thereof. Further, the connection cap 20 has an inside hole 22 formed at the center thereof to connect the body fluid inlet port 21 and the internal space of the suction container 10 with each other.

The drain tube 30 is coupled to the body fluid inlet port 21 and thus serves as a passage for inducing the body fluid discharged from the body of a human through the vacuum pressure of the suction container 10. A material or shape of the drain tube 30 is nearly the same as a general medical catheter, and the drain tube 30 has a clamp disposed at a given position to block an internal passage thereof.

The flow rate control member 40 is rotatably disposed in the inside hole 22 of the connection cap 20 and thus serves to control a flow rate of the body fluid introduced into the suction container 10 through the body fluid inlet port 21.

Lastly, the corrugated tube 50 contracts or expands in a longitudinal direction thereof according to the vacuum pressure of the suction container 10 and is made of soft silicone, rubber, or synthetic resin. The corrugated tube 50 is disposed on one side of the suction container 10 or on one side of the flow rate control member 40.

According to the present invention, as shown, the corrugated tube 50 is disposed on one side of the flow rate control member 40, and by means of a support ring 51, the corrugated tube 50 is fixed to an upper end periphery of the flow rate control member 40. The support ring 50 serves to reinforce the soft corrugated tube 50 to allow the soft corrugated tube 50 to be rigidly coupled to a top end periphery of the center hole 43 of the flow rate control member 40. Further, the corrugated tube 50 has a breathable waterproof film (not shown) disposed on an inlet thereof to block the backflow of the body fluid.

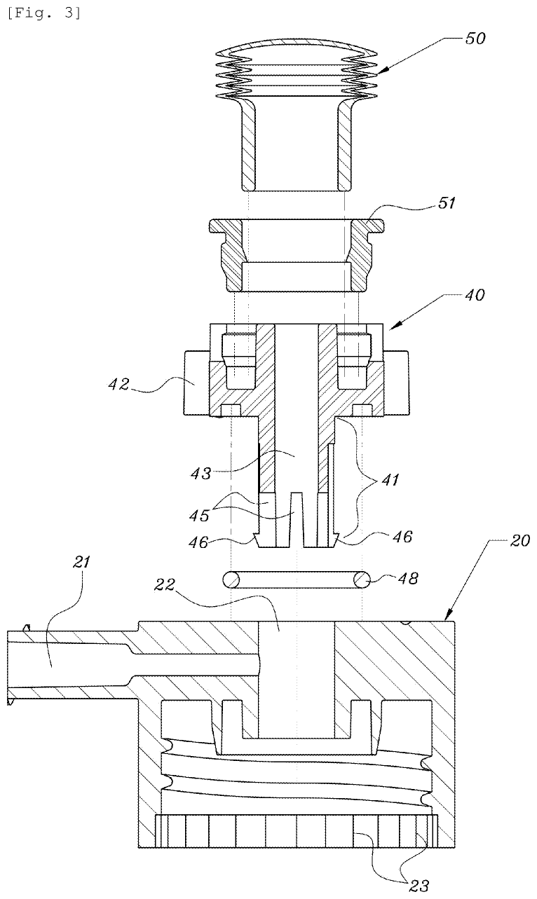

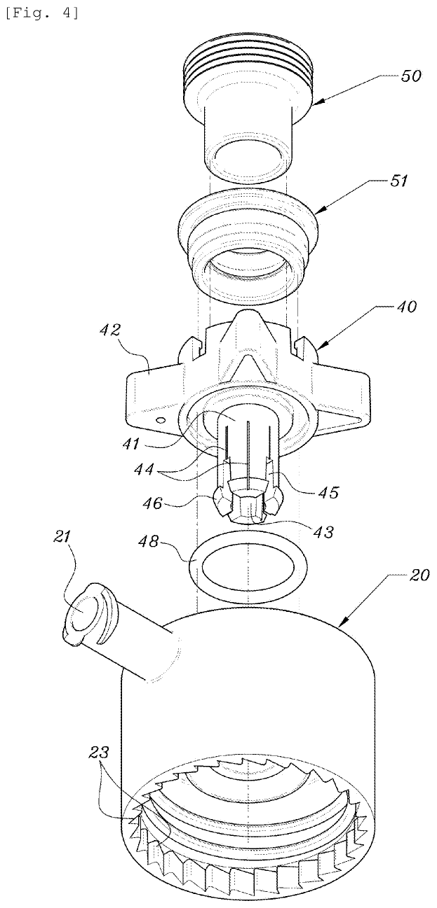

FIGS. 3 and 4 show main operating parts of the medical vacuum suction device according to a first embodiment of the present invention, wherein the flow rate control member 40 includes a cylinder 41 rotatably inserted into the inside hole 22 of the connection cap 20 and a handle 42 disposed on top of the cylinder to rotate the cylinder 41. Further, the flow rate control member 40 has a center hole 43 penetrated into the cylinder 41 and the handle 42 in a direction of a center axis thereof and a plurality of flow rate control grooves 44 formed on the outer peripheral surface of the cylinder 41 in parallel with each other.

The flow rate control grooves 44 are connected to the bottom end periphery of the cylinder 41 at positions connected to the body fluid inlet port 21 and provide different flow rates from each other. If the cylinder 41 rotates, accordingly, any one of the flow rate control grooves 44 is connected to the body fluid inlet port 21.

Further, the flow rate control member 40 has a plurality of elastic slits 45 incised along the lower end periphery of the cylinder 41 in a longitudinal direction thereof and circular locking projections 46 protruding from a bottom end periphery of the cylinder 41 in such a manner as to be lockedly coupled to the bottom end periphery of the inside hole 22. If the cylinder 41 pushes into the inside hole 22, accordingly, the circular locking projections 46 become narrow in space therebetween by means of the elasticity of the elastic slits 45 and thus pass through the inside hole 22. Next, the circular locking projections 46 are locked onto the bottom end periphery of the inside hole 22, so that the cylinder 41 is rotatably supported against the inside hole 22.

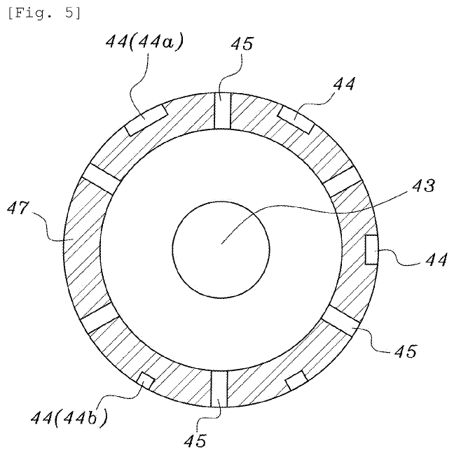

As shown in FIG. 5, the flow rate control grooves 44 are desirably formed along the outer periphery of the cylinder 41 in such a manner as to sequentially increase flow rates. At this time, a flow rate of a flow rate control groove 44a having the highest flow rate is the same as of the body fluid inlet port 21, and a flow passage cut-off wall 47 is disposed between the flow rate control groove 44a having the highest flow rate and a flow rate control groove 44b having the lowest flow rate to block the body fluid inlet port 21. Further, the flow rate control member 40 has an O-ring 48 disposed between the connection cap 20 and the flow rate control member 40 to prevent leakage from occurring in place and to maintain a sealed state therebetween.

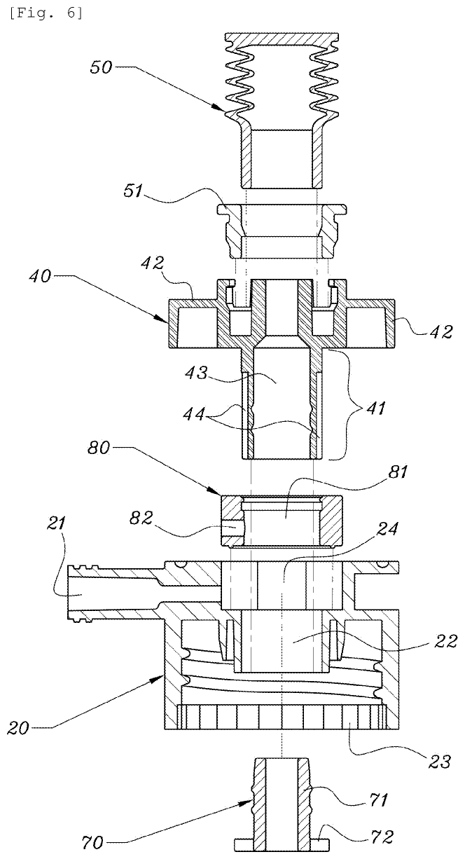

FIGS. 6 to 8 show main operating parts of the medical vacuum suction device according to a second embodiment of the present invention, wherein in the same manner as in the first embodiment of the present invention, the flow rate control member 40 includes a cylinder 41 rotatably inserted into the inside hole 22 of the connection cap 20 and a handle 42 disposed on top of the cylinder to rotate the cylinder 41. Further, the flow rate control member 40 has a center hole 43 penetrated into the cylinder 41 and the handle 42 in a direction of a center axis thereof and a plurality of flow rate control grooves 44 formed on the outer peripheral surface of the cylinder 41 in parallel with each other.

However, the flow rate control member 40 in the second embodiment of the present invention has no elastic slits 45 and circular locking projections 46 suggested in the first embodiment of the present invention, and instead of them, a fixing member 70 is coupled to the lower end periphery of the center hole 43 of the flow rate control member 40. The fixing member 70 includes an insertion tube 71 inserted into the inside of the center hole 43 and expanded wings 72 protruding circumferentially from the lower end periphery of the insertion tube 71 in such a manner as to be supported against a lower end projection of the inside hole 22.

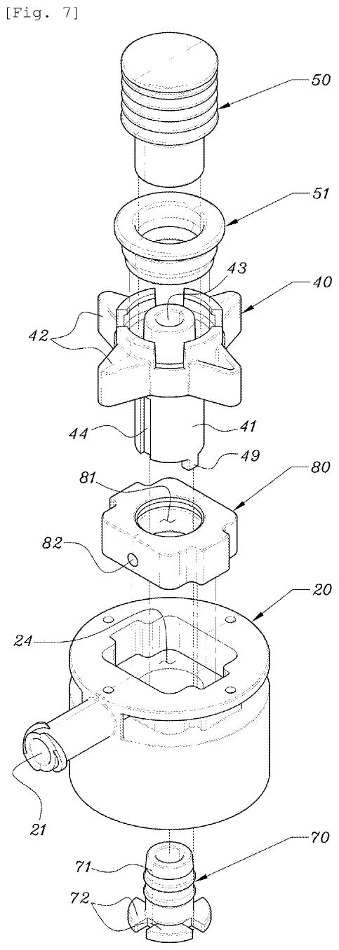

Through the fixing member 70, the cylinder 41 is rotatably supported in the inside hole 22 of the connection cap 20. At this time, the expanded wings 72 have to be located at positions where the passages of the flow rate control grooves 44 are not blocked. A reference numeral ‘49’ as not explained yet in FIG. 7, which protrudes from the bottom end periphery of the cylinder 41, indicates an alignment protrusion inserted between the expanded wings 72 to perform alignment so that the passages of the flow rate control grooves 44 are not blocked by means of the expanded wings 72.

Further, an expandable and contractible airtight member 80 is disposed between the connection cap 20 and the flow rate control member 40 to block the introduction of air. The airtight member 80 is made of an expandable and contractible material such as rubber, polyurethane, or the like and includes a cylinder insertion hole 81 for fittedly inserting the cylinder 41 and a body fluid passage 82 penetrated into the cylinder insertion hole 81 toward the body fluid inlet port 21.

Accordingly, the body fluid passage 82 is connected to any one of the flow rate control grooves 44 formed on the surface of the cylinder 41 inserted into the cylinder insertion hole 81. Further, the connection cap 20 has an airtight member accommodation portion 24 formed on a top surface thereof to fittedly accommodate the airtight member 80 therein.

The vacuum suction device of the present invention is configured to remove air from the interior of the suction container 10 through the body fluid inlet port 21 and then to block the body fluid inlet port 21 by means of the flow passage cut-off wall 47 to maintain the interior of the suction container 10 in the vacuum state, and in this state, the vacuum suction device is provided to the user. In a state where the drain tube 30 is inserted into an affected area of the body by the user, next, the cylinder 41 rotates through the handle 42 to control an amount of body fluid discharged.

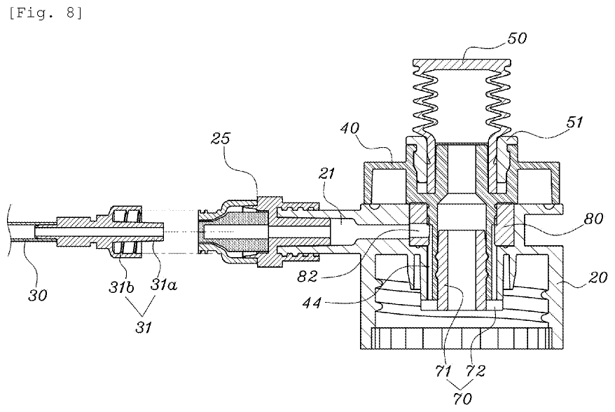

At this time, as shown in FIG. 8, the body fluid inlet hole 21 has a lure-lock connection port 25 connecting the drain tube 30 thereto, without having any spike, and the drain tube 30 has a luer connector 31 disposed on an end periphery thereof in such a manner as to be connected to the connection port 25. The luer connector 31 includes an insertion tube 31a inserted into a rubber stopper of the connection port 25 and a screw tube 31b screw-coupled to a male screw portion of the connection port 25.

Further, the user checks whether the interior of the suction container 10 is in the vacuum state or not through the contracted or expanded state of the corrugated tube 50. If the corrugated tube 50 is expanded, the vacuum state of the suction container 10 is released, so that it is impossible to use the suction container 10. Accordingly, as shown in FIG. 1, a one way exhaust valve 12 is disposed on one side of the suction container 10 to discharge the internal air of the suction container 10 through a general vacuum pump and thus to use the suction container 10 again in the case where the vacuum state of the suction container 10 is released.

Moreover, the suction container 10 is made of a generally opaque or semi-transparent material and desirably has a transparent window 13 disposed on one side thereof to see the content stored therein and scales indicated to measure an amount of the content. The transparent window 13 may be formed of a transparent tape attached to the surface of the suction container 10.

According to the present invention, an outer toothed rim 14 is formed along an outer periphery of the inlet 11 of the suction container 10, and an inner toothed rim 23 is formed along the inner lower end periphery of the connection cap 20 in such a manner as to be coupled to the outer toothed rim 14. The outer toothed rim 14 and the inner toothed rim 23 are formed in one way engaging with each other, and if they engage with each other, accordingly, the connection cap 20 is not open again. Accordingly, the outer toothed rim 14 and the inner toothed rim 23 serve as a safety cap for preventing the connection cap 20 from being open. Further, a hanging loop 15 is disposed on one side of the suction container 10 in such a manner as to hang on the user's cloth or belt, so that the suction container 10 can be portable.

According to another embodiment (not shown) of the present invention, the corrugated tube 50 may be disposed on one side of the suction container 10. In this case, the flow rate control member 40 does not have the center hole 43 as shown in FIGS. 3 and 4, and the interiors of the cylinder 41 and the handle 42 are closed.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A portable medical vacuum suction device comprising:
a suction container having an inlet formed on a top end periphery thereof and an internal space storing a body fluid discharged from the body of a human, the internal space being in a vacuum state;
a connection cap sealedly coupled to the inlet of the suction container and having a body fluid inlet port protruding from one side thereof and an inside hole formed at the center thereof to connect the body fluid inlet port and the internal space of the suction container with each other;
a drain tube coupled to the body fluid inlet port to induce the body fluid discharged from the body through a vacuum pressure of the suction container;
a flow rate control member rotatably disposed in the inside hole of the connection cap to control a flow rate of the body fluid introduced into the suction container through the body fluid inlet port; and
a soft corrugated tube disposed on one side of the suction container or the flow rate control member in such a manner as to contract or expand according to the vacuum pressure of the suction container.

2. The device according to claim 1, wherein the flow rate control member comprises: a cylinder rotatably inserted into the inside hole of the connection cap; a handle disposed on top of the cylinder; a center hole penetrated into the cylinder and the handle; a plurality of flow rate control grooves formed on the outer peripheral surface of the cylinder to control the flow rate of the body fluid introduced through the body fluid inlet port; a plurality of elastic slits formed along a lower end periphery of the cylinder; and circular locking projections protruding from a bottom end periphery of the cylinder in such a manner as to be lockedly coupled to a bottom end periphery of the inside hole.

3. The device according to claim 1, wherein the flow rate control member comprises: a cylinder rotatably inserted into the inside hole of the connection cap; a handle disposed on top of the cylinder; a center hole penetrated into the cylinder and the handle; and a plurality of flow rate control grooves formed on the outer peripheral surface of the cylinder to control the flow rate of the body fluid introduced through the body fluid inlet port, a fixing member is coupled to the lower end periphery of the center hole of the flow rate control member, the fixing member comprising: an insertion tube inserted into the inside of the center hole; and expanded wings protruding circumferentially from a lower end periphery of the insertion tube in such a manner as to be supported against a lower end projection of the inside hole, and the corrugated tube is disposed on an upper end periphery of the center hole of the flow rate control member by means of a support ring 1.

4. The device according to claim 2, wherein an expandable and contractible airtight member is disposed between the connection cap and the flow rate control member, the airtight member comprising a cylinder insertion hole for fittedly inserting the cylinder and a body fluid passage penetrated into the cylinder insertion hole toward the body fluid inlet port in such a manner as to be connected to any one of the flow rate control grooves formed on the surface of the cylinder, and the connection cap comprises an airtight member accommodation portion formed on a top surface thereof to fittedly accommodate the airtight member therein.

5. The device according to claim 2, wherein the flow rate control grooves are formed along the outer periphery of the cylinder in such a manner as to sequentially increase flow rates, and a flow passage cut-off wall is disposed between the flow rate control groove having the highest flow rate and the flow rate control groove having the lowest flow rate to block the body fluid inlet port.

6. The device according to claim 1, wherein the suction container has a one way exhaust valve disposed thereon to discharge the internal air thereof in one way.

7. The device according to claim 1, wherein the suction container is made of an opaque or semi-transparent material and has a transparent window disposed on one side thereof to see the content stored therein and scales indicated to measure an amount of the content.

8. The device according to claim 1, wherein the inlet of the suction container has a one way outer toothed rim formed along an outer periphery thereof, and the connection cap has an inner toothed rim formed along the inner lower end periphery thereof in such a manner as to be coupled to the outer toothed rim to prevent the connection cap from being open.

9. The device according to claim 1, wherein the corrugated tube has a breathable waterproof film disposed on an inlet thereof to block the backflow of the body fluid.

10. The device according to claim 3, wherein an expandable and contractible airtight member is disposed between the connection cap and the flow rate control member, the airtight member comprising a cylinder insertion hole for fittedly inserting the cylinder and a body fluid passage penetrated into the cylinder insertion hole toward the body fluid inlet port in such a manner as to be connected to any one of the flow rate control grooves formed on the surface of the cylinder, and the connection cap comprises an airtight member accommodation portion formed on a top surface thereof to fittedly accommodate the airtight member therein.

11. The device according to claim 3, wherein the flow rate control grooves are formed along the outer periphery of the cylinder in such a manner as to sequentially increase flow rates, and a flow passage cut-off wall is disposed between the flow rate control groove having the highest flow rate and the flow rate control groove having the lowest flow rate to block the body fluid inlet port.
